(19) United States
(12) Patent Application Publication
(10) Pub. No.: US 2007/0237821 A1
(43) Pub. Date: Oct. 11, 2007

(54) Nanogel-based contrast agents for optical molecular imaging

(75) Inventors: Jeffrey W. Leon, Rochester, NY (US); James R. Bennett, Rochester, NY (US); Tiecheng A. Qiao, Webster, NY (US); John W. Harder, Rochester, NY (US); Thomas H. Mourey, Rochester, NY (US); Gary L. Slater, Rochester, NY (US); Lijun Dai, Rochester, NY (US)

Correspondence Address: PATENT LEGAL STAFF;EASTMAN KODAK COMPANY, 343 STATE STREET, ROCHESTER, NY, 14650-2201, US

(73) Assignee: Eastman Kodak Company

(21) Appl. No.: 11/401,343

(22) Filed: Apr. 10, 2006

(51) Int. Cl. A61K 9/14

(52) U.S. Cl. 424/484

(57) ABSTRACT

The present invention relates to a nanogel comprising a polymer network of repetitive, crosslinked, ethylenically unsaturated monomers of Formula I:
(X)m-(Y)n-(Z)o Formula I wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional crosslinking monomer; m ranges from 50-90 mol %; n ranges from 2-30 mol %; and o range from 1-15 mol % and a method for preparing a nanogel comprising preparing a header composition of a mixture of monomers X, Y, and Z, and a first portion of initiators in water; preparing a reactor composition of a second portion initiators, surfactant, and water; bringing the reactor composition to the polymerization temperature; holding the reactor composition at the polymerization temperature, and adding the header composition to the reactor composition to form a nanogel of Formula I.

### CROSS REFERENCE TO RELATED APPLICATIONS

Reference is made to commonly assigned, co-pending U.S. patent applications:

Ser. No. ______ by Leon et al. (Docket 92267) filed of even date herewith entitled “LOADED LATEX OPTICAL MOLECULAR IMAGING PROBES”, and

Ser. No. ______ by Harder et al. (Docket 91687) filed of even date herewith entitled “FUNCTIONALIZED POLY(ETHYLENE GLYCOL)”, the disclosures of which are incorporated herein by reference.

### FIELD OF THE INVENTION

The present invention relates to injectable diagnostic agents for infrared medical imaging.

### BACKGROUND OF THE INVENTION

Recently, there has been intense interest focused upon developing nanoparticulate systems that are capable of carrying and delivering biological, pharmaceutical or diagnostic components within living systems. These systems are typically comprised of drugs, therapeutics, diagnostics, biocompatibilization functionalities, contrast agents, and targeting moieties attached to or contained within a nanoparticulate carrier. Work in this field has the goals of affording imaging and therapeutic agents with such profound advantages as greater circulatory lifetimes, higher specificity, lower toxicity and greater therapeutic effectiveness. Work in the field of nanoparticulate assemblies has promised to significantly improve the treatment of cancers and other life threatening diseases and may revolutionize their clinical diagnosis and treatment.

Certain nanoparticles were recently proposed as carriers for certain pharmaceutical agents. See, e.g., Sharma et al. Oncology Research 8, 281 (1996); Zobel et al. Antisense Nucl. Acid Drug Dev., 7:483 (1997); de Verdiere et al. Br. J. Cancer 76, 198 (1997); Hussein et al., Pharm. Res., 14, 613 (1997); Alyautdin et al. Pharm. Res. 14, 325 (1997); Hrkach et al., Biomaterials, 18, 27 (1997); Torchilin, J. Microencapsulation 15, 1 (1988); and literature cited therein. The nanoparticle chemistries provide for a wide spectrum of rigid polymer structures, which are suitable for the encapsulation of drugs, drug delivery and controlled release. Some major problems of these carriers include aggregation, colloidal instability under physiological conditions, low loading capacity, restricted control of the drug release kinetics, and synthetic preparations which are tedious and afford very low yields of product.

The size of the nanoparticulate assemblies is one major parameter determining their usefulness in biological compositions. After administration in the body, large particles are eliminated by the reticuloendothelial system and cannot be easily transported to the disease site (see, for example, Volkheimer, Pathologe 14:247 (1993); Kwon and Kataoka, Adv. Drug. Del. Rev. 16:295 (1995). Moghimi et al (Moghimi, S. M.; Hunter, A. C.; Murray, J. C. “Nanomedicine: Current Status and Future Prospects.” FASEB Journal 2005, 19, 311-330.) reports that particles larger than 100 nm are susceptible to clearance by interstitial macrophages while particles of 150 nm or larger are susceptible to accumulation in the liver. Also, the transport of large particles in the cell and intracellular delivery is limited or insignificant. See, e.g., Labhasetwar et al. Adv. Drug Del. Res. 24:63 (1997). It was demonstrated that an aggregated cationic species with a size from 500 nm to over 1 micron are ineffective in cell transfection. Large particles, particularly, those positively charged exhibit high toxicity in the body, in part due to adverse effects on liver and embolism. See e.g., Volkheimer, Pathologe 14:247 (1993); Khopade et al Pharmazie 51:558 (1996); Yamashita et al., Vet. Hum. Toxicol, 39:71 (1997).

Specific nanogels have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be more efficiently transferred across the arterial wall compared to larger size microparticles, see Labhasetwar et al., Adv. Drug Del. Res. 24:63 (1997). Without wishing to be bound by any particular theory it is also believed that because of high surface to volume ratio, the small size is essential for successful targeting of such particles using targeting molecules. Also, as nanogels occupy a hydrodynamic sphere which is mostly water, they can be functionalized with moieties of interest (biotargeting moieties, dyes, etc.) at much higher loading levels than solid particles.

It is also believed that maintaining the particle size distribution in the preferred range and thorough purification from larger particles is essential for the efficiency and safety of the nanogels. It is recognized that useful properties of the nanogels are determined solely by their size and structure and are independent of the method used for their preparation.

Due to their unique architecture, nanogels combine properties of cross-linked polymer gels and dispersed colloidal particles. They can be loaded with a variety of biological agents, including small molecules and polymers, at a very high biological agent to polymer network ratio. The immobilization of the biological agents in the nanogels is in the entire volume of the network rather than on its surface, and under certain conditions can be accompanied by the micro-collapse of the network providing for additional masking and protection of the biological agent. Aggregation of nanogels in-vivo have been identified as an impediment to the use of such systems (see Sun, X.; Rossin, R.; Turner, J. L.; Becker, M. L.; Joralemon, M. J.; Welch, M. J.; Wooley, K. L. “An Assessment of the Effects of Shell Cross-Linked Nanoparticle Size, Core Composition, and Surface PEGylation on in Vivo Biodistribution” Biomacromolecules 2005, 6, 2541-2554.)

U.S. Pat. No. 5,078,994 discloses a copolymer microparticle, prepared by emulsion polymerization, which is derived from at least about 5 weight percent of free carboxylic acid group-containing vinyl monomers, monomers which have a poly(alkylene oxide) appended thereto, oleophilic monomers and other nonionic hydrophilic monomers. Microgels containing these copolymers having a median water swollen diameter of about 0.01 to about 1.0 micrometer are disclosed. Pharmaceutical and diagnostic compositions are disclosed comprising a therapeutic or diagnostic agent and microgels comprising a copolymer derived from at least about 5 weight percent of non-esterified carboxylic acid group-containing vinyl monomers, oleophilic monomers and other nonionic hydrophilic monomers, with the proviso that when the median water swollen diameter of the microgels is 0.1 micrometer or greater, at least 5 weight percent of the monomers have a poly(alkylene oxide) appended thereto. Diagnostic and therapeutic methods are also disclosed wherein the microgels are substantially protein non-adsorbent and substantially refractory to phagocytosis. These particles, however, contain a large fraction of hydrophobic monomers and a low degree of PEGylation, and thus have inferior colloidal stability and biocompatibility.

US 2003/0211158 discloses novel microgels, microparticles, typically 0.1-10 microns in size, and related polymeric materials capable of delivering bioactive materials to cells for use as vaccines or therapeutic agents. The materials are made using a crosslinker molecule that contains a linkage cleavable under mild acidic conditions. The crosslinker molecule is exemplified by a bisacryloyl acetal crosslinker. The new materials have the common characteristic of being able to degrade by acid hydrolysis under conditions commonly found within the endosomal or lysosomal compartments of cells thereby releasing their payload within the cell. The materials can also be used for the delivery of therapeutics to the acidic regions of tumors and sites of inflammation. These particles, however, are of a large enough size range that uptake by the reticuloendothelial system can be expected to be a problem. In addition, the degree of PEGylation is low and in-vivo agglomeration has been identified as a problem (see Kwon, Y. J.; Standley, S. M.; Goh, S. L.; Frechet, J. M. J. Journal of Controlled Release 2005, 105, 199-212.)

U.S. Pat. No. 6,333,051 discloses copolymer networks having at least one cross-linked polyamine polymer fragment and at least one nonionic water-soluble polymer fragment, and compositions thereof, having at least one suitable biological agent. The invention relates to polymer technology, specifically polymer networks having at least one cross-linked polyamine polymer fragment at least one nonionic water-soluble polymer fragment, and compositions thereof. These nanogels, however, differ from those of this invention in that they are not based on ethylenically unsaturated backbone. In addition, the preparation of these nanogels is tedious and affords only small quantities.

The Journal of the American Chemical Society 124(51): 15198-15207 (“Polymeric Nanogels Produced via Inverse Microemulsion Polymerization as potential Gene and Antisense Delivery Agents”) describes crosslinked acrylate nanogels with quaternary amine functionalities and PEGDA crosslinker. The nanogels are approximately 40-200 nm in size. These nanogels, however, do not contain sufficient PEGylation and the preparation is tedious and only affords small quantities.

U.S. Pat. No. 5,874,111 discloses the preparation of highly monodispersed polymeric hydrophilic nanogels having a size of up to 100 nm, which may have drug substances encapsulated therein. The process comprises subjecting a mixture of an aqueous solution of a monomer or preformed polymer reverse micelles, a cross linking agent, initiator, and optionally, a drug or target substance to polymerization. The polymerized reaction product is dried for removal of solvent to obtain dried nanoparticles and surfactant employed in the process of preparing reverse micelles. The dry mass is dispersed in aqueous buffer and the surfactant and other toxic material are removed therefrom. This invention relates to a process for the preparation of highly monodispersed polymeric hydrophilic nanoparticles with or without target molecules encapsulated therein and having sizes of up to 100 nm and a high monodispersity. Again, these particles do not contain sufficient PEGylation to afford biocompatibility and the preparation is tedious.

Many authors have described the difficulty of making stable dispersions of surface modified particles. Achieving stability under physiological conditions (pH 7.4 and 137 mM NaCl) is yet even more difficult. Burke and Barret (Langmuir, 19, 3297 (2003)) describe the adsorption of the amine-containing polyelectrolyte, polyallylamine hydrochloride, onto 70-100 nm silica particles in the presence of salt. The authors state (p. 3299) “the concentration of NaCl in the solutions was maintained at 1.0 mM because higher salt concentrations lead to flocculation of the suspension”.

Siiman et al. U.S. Pat. No. 5,248,772 describes the preparation of colloidal metal particles having a cross-linked aminodextran coating with pendant amine groups attached thereto. The colloid is prepared at a very low concentration of solids 0.24% by weight, there is no indication of the final particle size, and there is no indication of the fraction of aminodextran directly bound to the surface of the colloid. Since the ratio of the weight of shell material (0.463 g) to the weight of core material (0.021 g) in example 2 is roughly 21:1, it appears likely that only a very small fraction of the aminodextran is bound to the surface of the colloid and that most remains free in solution. There is a problem in that this leads to a very small amount of active amine groups on the surface of the particle, and hence a very low useful biological, pharmaceutical or diagnostic components capacity for the described carrier particles in the colloids. There is an additional problem in that polymer not adsorbed to the particle surfaces may interfere with subsequent attachment or conjugation, of biological, pharmaceutical or diagnostic components. This reference, however, describes solid metal particles with a biocompatibilizing coating, which is fundamentally different from the hydrophilic nanogels of this invention.

U.S. Pat. No. 6,207,134 B1 describes particulate diagnostic contrast agents comprising magnetic or supermagnetic metal oxides and a polyionic coating agent. The coating agent can include “physiologically tolerable polymers” including amine-containing polymers. The contrast agents are said to have “improved stability and toxicity compared to the conventional particles” (col. 6, line 11-13). The authors state (Col. 4, line 15-16) that “not all the coating agent is deposited, it may be necessary to use 1.5-7, generally about two-fold excess . . . ” of the coating agent. The authors further show that only a small fraction of polymer adsorbs to the particles. For example, from FIG. 1 of '134, at 0.5 mg/mL polymer added only about 0.15 mg/mL adsorbs, or about 30%. The surface-modified particles of '134 are made by a conventional method involving simple mixing, sonication, centrifugation and filtration. Again, this describes polymer-coated solid metal particles, which are fundamentally different from the hydrophilic nanogels described herein.

### PROBLEM TO BE SOLVED

It would be desirable to produce nanogel for use as carriers for bioconjugation and targeted delivery which are stable so that they can be injected in vivo, especially intravascularly. Further, it is desirable that the nanogels for use as carriers be stable under physiological conditions (pH 7.4 and 137 mM NaCl). Still further, it is desirable that the particles avoid detection by the immune system. It is desirable to minimize the amount of polymeric material not adsorbed to the nanogel. In addition, nanogel probes are needed for Optical Molecular Imaging which are less than 100 nm in size, resist protein adsorption, have convenient attachment moieties for the attachment of biological targeting units, and contain emissive dyes that emit in the infrared (IR).

### SUMMARY OF THE INVENTION

The present invention relates to a nanogel comprising a water-compatible, swollen, branched polymer network of repetitive, crosslinked, ethylenically unsaturated monomers of Formula I:
(X)m-(Y)n-(Z)o Formula I
wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional crosslinking monomer; m ranges from 50-90 mol %; n ranges from 2-30 mol %; and o range from 1-15 mol %. The present invention also relates to a method for preparing a nanogel comprising preparing a header composition of a mixture of monomers X, Y, and Z, and a first portion of initiators in water, wherein X is a water-soluble monomer containing ionic or hydrogen bonding moieties, Y is a water-soluble macromonomer containing repetitive hydrophilic units bound to a polymerizeable ethylenically unsaturated group, and Z is a multifunctional crosslinking monomer; preparing a reactor composition of a second portion initiators, surfactant, and water sufficient to afford a composition of 1-10% w/w of monomers X, Y, and Z; bringing the reactor composition to the polymerization temperature; holding the reactor composition at the polymerization temperature for the duration of the reaction, and adding the header composition to the reactor composition over time to form a reaction mixture, wherein the nanogel comprises a water-compatible, swollen, branched polymer network of repetitive, crosslinked, ethylenically unsaturated monomers of Formula I:
(X)m-(Y)n-(Z)o Formula I
wherein m ranges from 50-90 mol %; n ranges from 2-30 mol %; and o range from 1-5 mol %.

### ADVANTAGEOUS EFFECT OF THE INVENTION

The present invention includes several advantages, not all of which are incorporated in a single embodiment. The materials of the present invention provide a medium for high loading levels of dyes, are stable within a broad window of conditions, are easy to prepare, and demonstrate high biological compatibility.

### BRIEF DESCRIPTION OF THE DRAWINGS

FIG. 1 illustrates a normalized absorbance spectra of exemplified dye-loaded Nanogel 1 and 0.0125 mg/ml Dye 1 in PBS buffer.

### DETAILED DESCRIPTION OF THE INVENTION

The present invention relates to a nanogel comprising a water-compatible, swollen, branched polymer network of repetitive, crosslinked, ethylenically unsaturated monomers of a particular formula.

Specific nanogels have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be more efficiently transferred across the arterial wall compared to larger size microparticles, see Labhasetwar et al., Adv. Drug Del. Res. 24:63 (1997). Without wishing to be bound by any particular theory it is also believed that because of high surface to volume ratio, the small size is essential for successful targeting of such particles using targeting molecules. Also, as nanogels occupy a hydrodynamic sphere which is mostly water, they can be functionalized with moieties of interest (biotargeting moieties, dyes, etc.) at much higher loading levels than solid particles.

It is also believed that maintaining the particle size distribution in the preferred range and thorough purification from larger particles is essential for the efficiency and safety of the nanogel. It is recognized that useful properties of the nanogels are determined solely by their size and structure and are independent of the method used for their preparation. Therefore, this invention is not limited to a certain synthesis or purification procedures, but rather encompasses new and novel chemical entities useful in biological agent compositions.

Nanogels of the current invention are soluble, highly stable and do not aggregate across a wide window of physiological and experimental conditions. The loading capacity of nanogels can be as high as several grams or several dozen grams per one gram of the polymer network. This is much higher compared to the loading capacity achieved with nanoparticles. See, Labhasetwar et al., Adv. Drug Del. Res., 24:63 (1997). In contrast to conventional drug delivery particles such as solid nanoparticles (which often have to be prepared in the presence of the biological agent) the polymer network may be loaded with the biological agent after the network its synthesized. This greatly simplifies the preparation and use of the biological agent composition of this invention and permits using batches of nanogel with many different biological agents and compositions.

Whenever used in the specification the terms set forth shall have the following meaning:

The term “nanogel” refers to a swollen, contiguous, crosslinked polymer network in the size range of 5-100 nanometers through which a through-bond path can be traced between any two atoms (not including counterions).

The term nanoparticle or nanoparticulate refers to a particle with a size of less than 100 nm.

The term “colloid” refers to a mixture of small particulates dispersed in a liquid, such as water.

The term “biocompatible” means that a composition does not disrupt the normal function of the bio-system into which it is introduced. Typically, a biocompatible composition will be compatible with blood and does not otherwise cause an adverse reaction in the body. For example, to be biocompatible, the material should not be toxic, immunogenic or thrombogenic.

The term “biodegradable” means that the material can be degraded either enzymatically or hydrolytically under physiological conditions to smaller molecules that can be eliminated from the body through normal processes.

The term “brush polymer” refers to a polymer in which relatively uniform, macromolecular “arms,” each of a molecular weight of 400 Daltons or greater eminate from a contiguous polymeric backbone, wherein the arms are each attached to the backbone at only one of their two possible ends and the distribution of the arms along the backbone is relatively uniform.

The term “swollen” refers to the solvated state which the polymer associates with the solvent molecules rather than with each other, thereby expanding the total volume occupied by the single polymer molecule.

The term “water compatible” refers to a material which exists in a swollen state in water over the temperature range of 5-80° C.

The nanogel is a stable solution or dispersion. The dispersion is said to be stable if the solid particulates do not aggregate, as determined by particle size measurement, and settle from the dispersion, usually for a period of hours, preferably weeks to months. Terms describing instability include aggregation, agglomeration, flocculation, gelation and settling. Significant growth of mean particle size to diameters greater than about three times the core diameter, and visible settling of the dispersion within one day of its preparation is indicative of an unstable dispersion. Preferably the nanogel is stable at 20-35° C. in 0.137M NaCl at pH 7.4. Most preferably the nanogel is stable in 0.8 M NaCl.

The nanogels of this invention are substantially non-adsorbent to serum proteins. For in-vivo applications, it is desirable that a nanoparticle will have a long circulation lifetime. The adsorption of serum protein entities onto the surface of a nanoparticle (opsonization) will usually preclude their removal from circulation, often by uptake by macrophages or monocytes. Even in the case that they are not removed from circulation, nonspecific binding of proteins to the surface of nanoparticles may foul the surface and shield desirable functionalities, such as biotargeting moieties. Examples of serum proteins include various subclasses of immunoglobulins, complement proteins, apolipoproteins, von Willebrand factor, thrombospondin, fibronectin, mannose-binding proteins, and plasma proteins, such as serum albumins. For the purpose of this invention, a nanogel may be considered to be substantially serum protein non-adsorbent if it is non-adsorbent to bovine serum albumin (BSA), a model serum protein. This property can be tested by combining the nanogel and BSA and performing size exclusion chromatography in PBS buffer. If the nanogel is non-adsorbent to the BSA, then the retention volume of the BSA will be no different than that of the BSA itself, and the overall chromatographic curve shape will be equal to the combination of those of the individual components (BSA and nanogel).

In a preferred embodiment, the nanogel is made of a water-compatible, swollen, branched polymer or macromer, wherein macromer denotes a macromonomer, of repetitive, crosslinked, ethylenically unsaturated monomers of Formula I:
(X)m-(Y)n-(Z)o Formula I
In Formula I, X is a highly hydrophilic monomer containing ionic moieties or exchangeable proton-containing moieties; Y is a water-soluble macromonomer containing repetitive hydrophilic units bound to a polymerizeable ethylenically unsaturated group; and Z is a multifunctional crosslinking monomer. Exchangeable proton-containing moieties may include alcohols, primary and secondary amines, primary amides, secondary amides, carboxylic acids, carbamates, imides, ureas, phosphonic acids, sulfonic acids, sulfinic acids, or any other unit which contains a heteroatom (N,O,S,P)-hydrogen bond.

“Highly hydrophilic monomers” are defined as having calculated log P values of 0.4 or less. The Log P value is the logarithm of the octanol-water partition coefficient of the compounds. The octanol/water partition coefficient (P) of a compound is the ratio of the amount of material that dissolves in the octanol phase divided by the concentration in the aqueous phase at equilibrium. Log P is often used to describe the relative tendency of a molecule to favor an oil (octanol) or water phase (see Leo and Hansch, “Substituent Constants for Correlation Analysis in Chemistry and Biology,” Wiley, New York, 1979, and in Leo, Hansch, and Elkins, Chem. Rev., 6, 525, (1971)). It is a measure of how hydrophobic or hydrophilic the molecule is. It can be difficult to measure partition coefficients for monomers; however, methods have been developed for calculating a log P from a compounds molecular structure. For example, the KOWWIN© program Version 1.6, developed by the SYRACUSE RESEARCH CORPORATION, Environmental Science Center, 6225 Running Ridge Road, North Syracuse, N.Y. 13212-2510 is such a program.

In this invention, m may range from 50-90 mol %, preferably from 60-80 mol %. Also, n may range from 2-30 mol %, preferably from 10-20 mol % and o may reange from 1-15 mol %, preferably from 2-9 mol %.

X is a water-soluble monomer containing ionic or exchangeable proton-containing moieties. Especially useful highly hydrophilic “X” monomers may be described by the formula below 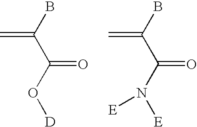
Wherein B is H or CH₃, and D may each be H, a nonionic unit with a hydrogen bonding moiety and containing no more than three carbons, or an ionic unit comprised of up to six carbons. E may have the composition as B except that additionally E may be CH₃. X may be, but is not necessarily limited to methacrylic acid, acrylic acid, acrylamide, methacrylamide, aminopropyl methacrylamide hydrochloride, sulfopropyl methacrylate, hydroxyethyl acrylate or hydroxyethyl methacrylate, N-methyl acrylamide, or N,N-dimethylacrylamide

Y is a water-soluble macromonomer with a molecular weight of between 200 and 20,000, preferably between 400 and 10000 and is comprised of repetitive water-soluble units. Preferably Y is a poly(ethylene glycol) macromonomer such as a poly(ethylene glycol)acrylate, poly(ethylene glycol)methacrylate, N-poly(ethylene glycol)acrylamide, N-poly(ethylene glycol)methacrylamide, or a poly(ethylene glycol) macromonomer with a styrenic terminus.

Crosslinking monomer Z may be highly hydrophilic or organic-soluble crosslinker such asmethylenebisacrylamide, N,N′-(1,2-dihydroxyethylene)bisacrylamide, methylenebismethacrylamide divinylbenzene, ethylene glycol dimethacrylate, Preferably, the crosslinking monomer is difunctional, trifunctional, or tetrafunctional and has a molecular weight of less than 300 Daltons. At least 90% of the total monomers should be highly hydrophilic or water-soluble monomers. The remaining 10% may comprise monomers that are organic-soluble or are not highly hydrophilic.

The particle size(s) of the nanogel may be characterized by a number of methods, or combination of methods, including, light-scattering methods, sedimentation methods such as analytical ultracentrifugation, hydrodynamic separation methods such as field flow fractionation and size exclusion chromatography, and electron microscopy. The nanogels in the examples were characterized primarily using light-scattering methods. Light-scattering methods can be used to obtain information regarding volume median particle diameter, the particle size number and volume distribution of nanogels, standard deviation of the distribution(s) and the distribution width.

The nanogel may have a volume average hydrodynamic volume median diameter of between 10 and 100, preferably 10 to 50 nm as determined by quasi-elastic light scattering in phosphate buffered saline (137 mM NaCl, 2.7 mM KCl, 10 mM Na₂HPO₄, 2 mM KH₂PO₄ at pH 7.4.). Hydrodynamic diameter refers to the diameter of the equivalent sphere of the polymer and its associated solvent as determined by quasi-elastic light scattering.

The nanogel may also have a weight average molecular weight of from 15,000 to 6,000,000, preferably, from 80,000 to 800,000 and most preferably from 100,000 to 400,000 as measured by static light scattering or by size exclusion chromatography. The weight average degree of polymerization of the nanogel may be from 50 to 86,000, preferably from 100 to 1500. The degree of polymerization may be calculated from the weight average molecular weight and from the molecular weights and mole fractions of the component monomers. The mole fractions of the component monomers may be determined from the recipe from which the nanogel was prepared or by any other suitable analytical method for determining polymer composition (NMR, titrations, etc). The nanogel may have a φ₂ parameter between 0.01 and 0.30, in water, preferably from 0.02 to 0.20. The φ₂ parameter is a measure of the density of the nanogel within the hydrodynamic sphere. It is calculated by the following equation

$${ϕ}_{2} = \frac{{M}_{w}}{{N}_{A}} {( \frac{4}{3} π {R}_{h}^{3} )}^{- 1}$$

wherein M_{w} is the weight average molecular weight as determined by static light scattering or by size exclusion chromatography, Rₕ is the hydrodynamic radius as measured by quasi-elastic light scattering or by other suitable methods, and NA is Avogadro's number.

The intrinsic viscosity is between 0.40 dL/g and 0.85 dL/g as measured in 1,1,1,2,2,2-hexafluoro-2-propanol (HFIP). The intrinsic viscosity is the viscosity of a polymer in solution at infinite dilution. It may be determined by capillary tube viscometry methods, such as those described in Principles of Colloid and Surface Chemistry (Paul C. Heimenz and Raj Rajahgopalan, Marcel Dekker Inc, New York 1997) or in Colloidal Systems and Interfaces (Sydney Ross and Ian Morrison, John Wiley and Sons, New York, 1988).

As the nanogels may be utilized under a wide range of chemical conditions, it is advantageous that the nanogels do not undergo a sharp decrease in size with increasing temperature. Many known nanogel and microgel materials will undergo a sharp morphological change with increasing temperature in which the material collapses, undergoing a sometimes drastic change in volume. Such transitions are not advantageous in drug delivery and imaging applications, as such sharp morphological changes may perturb the disposition of or result in the rearrangement of the surface groups, payload, and morphology of the nanogel composition. This can be especially disadvantageous when this transition occurs at or near physiological temperatures. The nanogels of this invention, thus, will show either a small change (<25%) or a net increase of hydrodynamic diameter upon raising the temperature from 25° C. to 80° C.

The present nanogels can be useful as a carrier for carrying a biological, pharmaceutical or diagnostic component. Specifically, the nanogel used as a carrier does not necessarily encapsulate a specific therapeutic or an imaging component, but rather serve as a carrier for the biological, pharmaceutical or diagnostic components. Biological, pharmaceutical or diagnostic components such as therapeutic agents, diagnostic agents, dyes or radiographic contrast agents. The term “diagnostic agent” includes components that can act as contrast agents and thereby produce a detectable indicating signal in the host mammal. The detectable indicating signal may be gamma-emitting, radioactive, echogenic, fluoroscopic or physiological signals, or the like. The term biomedical agent, as used herein, includes biologically active substances which are effective in the treatment of a physiological disorder, pharmaceuticals, enzymes, hormones, steroids, recombinant products, and the like. Exemplary therapeutic agents are antibiotics, thrombolytic enzymes such as urokinase or streptokinase, insulin, growth hormone, chemotherapeutics such as adriamycin and antiviral agents such as interferon and acyclovir. Upon enzymatic degradation, such as by a protease or a hydrolase, the therapeutic agents can be released over a period of time.

Included within the scope of the invention are compositions comprising the polymer networks of the current invention and a suitable targeting molecule. As used herein, the term “targeting molecule” refers to any molecule, atom, or ion linked to the polymer networks of the current invention that enhance binding, transport, accumulation, residence time, bioavailability or modify biological activity of the polymer networks or biologically active compositions of the current invention in the body or cell. The targeting molecule will frequently comprise an antibody, fragment of antibody or chimeric antibody molecules typically with specificity for a certain cell surface antigen. It could also be, for instance, a hormone having a specific interaction with a cell surface receptor, or a drug having a cell surface receptor. For example, glycolipids could serve to target a polysaccharide receptor. It could also be, for instance, enzymes, lectins, and polysaccharides. Low molecular mass ligands, such as folic acid and derivatives thereof are also useful in the context of the current invention. The targeting molecules can also be polynucleotide, polypeptide, peptidomimetic, carbohydrates including polysaccharides, derivatives thereof or other chemical entities obtained by means of combinatorial chemistry and biology. Targeting molecules can be used to facilitate intracellular transport of the nanogels of the invention, for instance transport to the nucleus, by using, for example, fusogenic peptides as targeting molecules described by Soukchareun et al., Bioconjugate Chem., 6, 43, (1995) or Arar et al., Bioconjugate Chem., 6, 43 (1995), caryotypic peptides, or other biospecific groups providing site-directed transport into a cell (in particular, exit from endosomic compartments into cytoplasm, or delivery to the nucleus).

The described composition can further comprise a biological, pharmaceutical or diagnostic component that includes a targeting moiety that recognizes the specific target cell. Recognition and binding of a cell surface receptor through a targeting moiety associated with a described nanogel used as a carrier can be a feature of the described compositions. For purposes of the present invention, a compound carried by the nanogel may be referred to as a “carried” compound. For example, the biological, pharmaceutical or diagnostic component that includes a targeting moiety that recognizes the specific target cell described above is a “carried” compound. This feature takes advantage of the understanding that a cell surface binding event is often the initiating step in a cellular cascade leading to a range of events, notably receptor-mediated endocytosis. The term “Receptor Mediated Endocytosis” (“RME”) generally describes a mechanism by which, catalyzed by the binding of a ligand to a receptor disposed on the surface of a cell, a receptor-bound ligand is internalized within a cell. Many proteins and other structures enter cells via receptor mediated endocytosis, including insulin, epidermal growth factor, growth hormone, thyroid stimulating hormone, nerve growth factor, calcitonin, glucagon and many others.

Receptor Mediated Endocytosis affords a convenient mechanism for transporting a described nanogel, possibly containing other biological, pharmaceutical or diagnostic components, to the interior of a cell. In RME, the binding of a ligand by a receptor disposed on the surface of a cell can initiate an intracellular signal, which can include an endocytosis response. Thus, a nanogel used as a carrier with an associated targeting moiety, can bind on the surface of a cell and subsequently be invaginated and internalized within the cell. A representative, but non-limiting, list of moieties that can be employed as targeting agents useful with the present compositions includes proteins, peptides, aptomers, small organic molecules, toxins, diptheria toxin, pseudomonas toxin, cholera toxin, ricin, concanavalin A, Rous sarcoma virus, Semliki forest virus, vesicular stomatitis virus, adenovirus, transferrin, low density lipoprotein, transcobalamin, yolk proteins, epidermal growth factor, growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysacchlorides. Homologs or fragments of the presented moieties can also be employed. These targeting moieties can be associated with a nanogel and be used to direct the nanogel to a target cell, where it can subsequently be internalized. There is no requirement that the entire moiety be used as a targeting moiety. Smaller fragments of these moieties known to interact with a specific receptor or other structure can also be used as a targeting moiety.

An antibody or an antibody fragment represents a class of most universally used targeting moiety that can be utilized to enhance the uptake of nanogels into a cell. Antibodies may be prepared by any of a variety of techniques known to those of ordinary skill in the art. See, e.g., Harlow and Lane, Antibodies: A Laboratory Manual, Cold Spring Harbor Laboratory, 1988. Antibodies can be produced by cell culture techniques, including the generation of monoclonal antibodies or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described (Winter et al. (1991) Nature 349:293-299; Lobuglio et al. (1989) Proc. Nat. Acad. Sci. USA 86:4220-4224. These “humanized” molecules are designed to minimize unwanted immunological response toward rodent antihuman antibody molecules that limits the duration and effectiveness of therapeutic applications of those moieties in human recipients.

Vitamins and other essential minerals and nutrients can be utilized as targeting moiety to enhance the uptake of nanogel by a cell. In particular, a vitamin ligand can be selected from the group consisting of folate, folate receptor-binding analogs of folate, and other folate receptor-binding ligands, biotin, biotin receptor-binding analogs of biotin and other biotin receptor-binding ligands, riboflavin, riboflavin receptor-binding analogs of riboflavin and other riboflavin receptor-binding ligands, and thiamin, thiamin receptor-binding analogs of thiamin and other thiamin receptor-binding ligands. Additional nutrients believed to trigger receptor mediated endocytosis, and thus also having application in accordance with the presently disclosed method, are carnitine, inositol, lipoic acid, niacin, pantothenic acid, pyridoxal, and ascorbic acid, and the lipid soluble vitamins A, D, E and K. Furthermore, any of the “immunoliposomes” (liposomes having an antibody linked to the surface of the liposome) described in the prior art are suitable for use with the described compositions.

Since not all natural cell membranes possess biologically active biotin or folate receptors, use of the described compositions in-vitro on a particular cell line can involve altering or otherwise modifying that cell line first to ensure the presence of biologically active biotin or folate receptors. Thus, the number of biotin or folate receptors on a cell membrane can be increased by growing a cell line on biotin or folate deficient substrates to promote biotin and folate receptor production, or by expression of an inserted foreign gene for the protein or apoprotein corresponding to the biotin or folate receptor.

RME is not the exclusive method by which the described nanogel can be translocated into a cell. Other methods of uptake that can be exploited by attaching the appropriate entity to a nanogel include the advantageous use of membrane pores. Phagocytotic and pinocytotic mechanisms also offer advantageous mechanisms by which a nanogel can be internalized inside a cell.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

A cell surface recognition sequence is not a requirement. Thus, although a cell surface receptor targeting moiety can be useful for targeting a given cell type, or for inducing the association of a described nanogel with a cell surface, there is no requirement that a cell surface receptor targeting moiety be present on the surface of a nanogel.

To assemble the biological, pharmaceutical or diagnostic components to a described nanogel used as a carrier, the components can be associated with the nanogel carrier through a linkage. By “associated with”, it is meant that the component is carried by the nanogel. The component can be dissolved and incorporated in the nanogel non-covalently.

Generally, any manner of forming a linkage between a biological, pharmaceutical or diagnostic component of interest and a nanogel used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanogel used as a carrier through the formation of amide, ester or imino bonds between acid, aldehyde, hydroxy, amino, or hydrazo groups on the respective components of the complex. Art-recognized biologically labile covalent linkages such as imino bonds and so-called “active” esters having the linkage —COOCH, —O—O— or —COOCH are preferred. The biological, pharmaceutical or diagnostic component of interest may be attached to the pre-formed nanogel or alternately the component of interest may be pre-attached to a polymerizeable unit and polymerized directly into the nanogel during the nanogel preparation. Hydrogen bonding, e.g., that occurring between complementary strands of nucleic acids, can also be used for linkage formation.

In a preferred embodiment of this invention, the biological, pharmaceutical or diagnostic component of interest is attached to the nanogel by reaction with a reactive chemical unit at the terminus of the highly hydrophilic macromonomer units. Preferably this reactive chemical unit is a carboxylic acid, amine, or activated ester. Most preferably, this attachment occurs via a linking polymer.

The linking polymer may be used in both the acylation and alkylation approaches and is compatible with aqueous and organic solvent systems, so that there is more flexibility in reacting with useful groups and the desired products are more stable in an aqueous environment, such as a physiological environment. The linking polymer has a poly(ethylene glycol) backbone structure which contains at least two reactive groups, one at each end. The poly(ethylene glycol) macromonomer backbone contains a radical polymerizeable group at one end. This group can be, but is not necessarily limited to a methacrylate, acrylate, acrylamide, methacrylamide, styrenic, allyl, vinyl, maleimide, or maleate ester. The poly(ethylene glycol) macromonomer backbone additionally contains a reactive chemical functionality at the other end which can serve as an attachment point for other chemical units, such as quenchers or antibodies. This chemical functionality may be, but is not limited to thiols, carboxylic acids, primary or secondary amines, vinylsulfonyls, aldehydes, epoxies, hydrazides, succinimidyl esters, maleimides, a-halo carbonyl moieties (such as iodoacetyls), isocyanates, isothiocyanates, and aziridines. Preferably, these functionalities will be carboxylic acids, primary amines, maleimides, vinylsulfonyls, or secondary amines. Most preferably, one of the reactive groups is an acrylate, cyanoacrylate, or a methacrylate which is useful for forming nanogels and latexes and reacting with thiols through Michael addition. The other reactive group is useful for conjugation to contrast agents, dyes, proteins, amino acids, peptides, antibodies, bioligands, therapeutic agents and enzyme inhibitors. The linking polymer may be branched or unbranched. Preferably, for therapeutic use of the end-product preparation, the linking polymer will be pharmaceutically acceptable. The poly(ethylene glycol) macromonomer may have a molecular weight of between 300 and 10,000, preferably between 500 and 5000.

A particularly preferred water-soluble linking polymer for use herein is a poly(ethylene glycol) derivative of Formula I. The poly(ethylene glycol) (PEG) backbone of the linking polymer is a hydrophilic, biocompatible and non-toxic polymer of general formula H(OCH (2)CH (2)) (n)OH, wherein n>4. 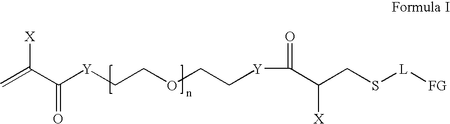
wherein X═CH3 or H, Y═O, NR, or S, L is a linking group or spacer, FG is a functional group, n is greater than 4 and less than 1000. Most preferably, X═CH3, Y═O, NR, L is alkyl or aryl and FG is NH2 or COOH, and n is between 6 and 500 or between 10 and 200. Most preferably, n=16.

The following is a list of preferred linking polymers, but is not intended to an exhaustive and complete list of all linking polymers according to the present invention: 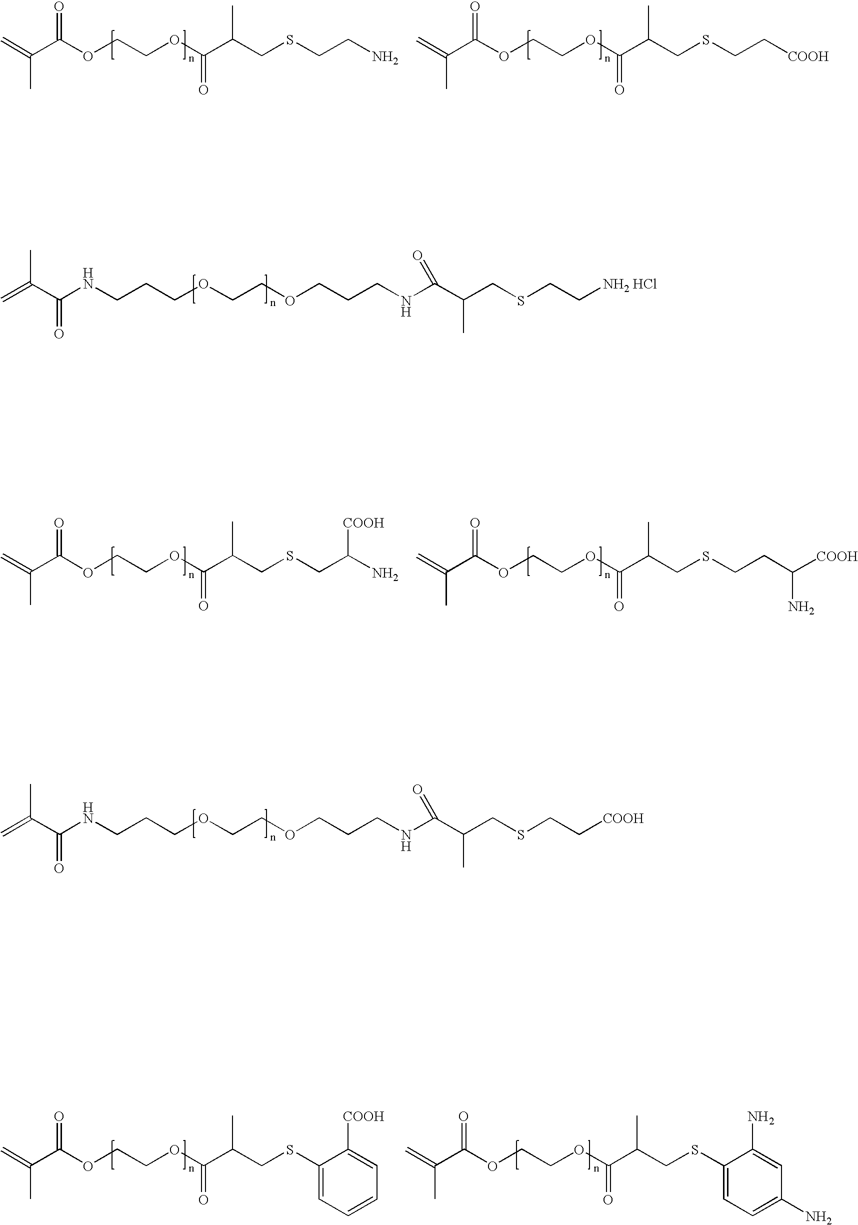 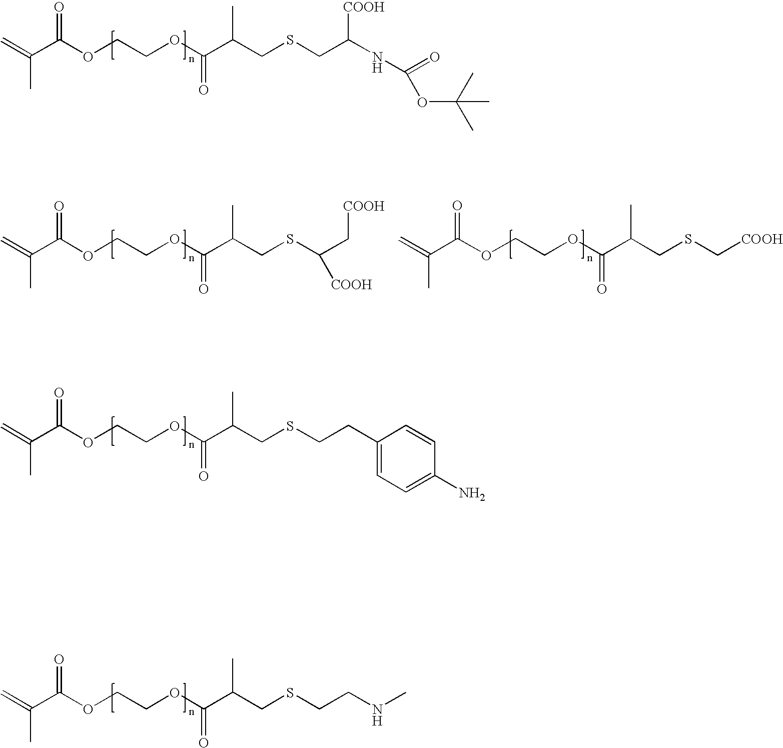
Any of the linking polymers discussed above are also useful as the Y monomer of the nanogel according to the present invention.

After a sufficiently pure nanogel, preferably comprising a nanogel with a biological, pharmaceutical or diagnostic component, has been prepared, it might be desirable to prepare the nanogel in a pharmaceutical composition that can be administered to a subject or sample. Preferred administration techniques include parenteral administration, intravenous administration and infusion directly into any desired target tissue, including but not limited to a solid tumor or other neoplastic tissue. Purification can be achieved by employing a final purification step, which dissolves the nanogel in a medium comprising a suitable pharmaceutical composition. Suitable pharmaceutical compositions generally comprise an amount of the desired nanogel with active agent in accordance with the dosage information (which is determined on a case-by-case basis). The described nanogels are admixed with an acceptable pharmaceutical diluent or excipient, such as a sterile aqueous solution, to give an appropriate final concentration. Such formulations can typically include buffers such as phosphate buffered saline (PBS), or additional additives such as pharmaceutical excipients, stabilizing agents such as BSA or HSA, or salts such as sodium chloride.

For parenteral administration it is generally desirable to further render such compositions pharmaceutically acceptable by insuring their sterility, non-immunogenicity and non-pyrogenicity. Such techniques are generally well known in the art. Moreover, for human administration, preparations should meet sterility, pyrogenicity, general safety and purity standards as required by FDA Office of Biological Standards. When the described nanogel composition is being introduced into cells suspended in a cell culture, it is sufficient to incubate the cells together with the nanogel in an appropriate growth media, for example Luria broth (LB) or a suitable cell culture medium. Although other introduction methods are possible, these introduction treatments are preferable and can be performed without regard for the entities present on the surface of a nanogel used as a carrier.

The nanogels of this invention may be prepared via a solution polymerization with continuous addition of monomer. This method comprises preparing a “header” composition of a mixture of all of the monomers, a first portion of the initiators and optional surfactant in water, preparing a “reactor” composition of a second portion of the initiators and surfactant and water sufficient to afford a composition of 1-10% w/w of total monomers, bringing said “reactor” composition to the polymerization temperature, holding said “reactor” composition at said polymerization temperature for the duration of the reaction, and adding said “header” composition to said “reactor” composition over time to form a reaction mixture. Further, the reaction mixture may be heated for up to 48 hours and the reacted mixture may further be purified by dialysis, ultrafiltration, diafiltration, or treatment with ion exchange resins.

The “header” composition is prepared consisting of a mixture of all of the monomers, 0-100% of the initiators and 0-100% of the surfactant (if surfactant is used), 0-100% of the water. The monomer mixture comprises 50-90 mol % of one or more “Type X” monomers, (preferably from 60-80 mol %), 2-30 mol % of a “Type Y” monomer (preferably from 10-20 mol %), and 1-20 mol % of a “type Z” monomer (preferably from 11-15 mol %. Type X, Y, and Z monomers are described in an earlier section of this document.

The initiator may be any of the common water-soluble polymerization initiators known in the art of addition polymerization. These include, but are not restricted to azo compounds, such as 4,4′-azobis(4-cyanopentanoic acid), and 2,2′-azobis(2-amidinopropane)dihydrochloride, 2,2′-azobis(N,N′-dimethyleneisobutyramidine) and its dihydrochloride salt, 2,2′-azobis[2-methyl-N-(2-hydroxyethyl)propionamide], water-soluble peroxides, hydroperoxides, and peracids such as peracetic acid and hydrogen peroxide, persulfate salts such as potassium, sodium and ammonium persulfate, disulfides, tetrazenes, and redox initiator systems such as H₂O₂/Fe²⁺, persulfate/bisulfite, oxalic acid/Mn³⁺, thiourea/Fe³⁺. Preferably a water-soluble azo initiator is used. If a redox or two component initiator is used, one component will typically be included in the header and the other component will be included in the reactor, such that free radicals are steadily generated as the two mixtures are combined. Alternately, water-soluble photoinitiators can be used in combination with an irradiation source.

Surfactants which can be used in this invention can be anionic, cationic, zwitterionic, neutral, low molecular weight, macromolecular, synthetic, or extracted from or derived form natural sources. There exist a tremendous number of known surfactants. Good reference sources for surfactants are the Surfactant Handbook (GPO: Washington, D.C., 1971) and McCutcheon's Emulsifiers and Detergents (Manufacturing Confectioner Publishing Company: Glen Rock, 1992). Some examples include, but are not necessarily limited to: sodium dodecylsulfate, sodium dodecylbenzenesulfonate, sulfosuccinate esters, such as those sold under the AEROSOL® trade name, ethoxylated alkylphenols, such as TRITON® X-100 and TRITON® X-705, ethoxylated alkylphenol sulfates, such as RHODAPEX® CO-436, phosphate ester surfactants such as GAFAC® RE-90, hexadecyltrimethylammonium bromide, cetylpyridinium chloride, polyoxyethylenated long-chain amines and their quaternized derivatives, alkanolamine condensates, polyethylene oxide-co-polypropylene oxide block copolymers, such as those sold under the PLURONIC® and TECTRONIC® trade names, N-alkylbetaines, N-alkyl amine oxides, and sulfonated diphenyl ethers, such as those sold under the Dowfax® tradename.

The “reactor” composition is prepared consisting of the remaining initiators and surfactant and water sufficient to afford a composition of 1-10% w/w of total monomers.

The reactor composition is brought to the polymerization temperature and held there for the duration of the reaction. This is the temperature at which the polymerization initiator is known to be sufficiently active. For example, using AIBN or potassium persulfate or 4,4′-azobis(4-cyanopentanoic acid), 60-80° C. is usually sufficient. For the persulfate/bisulfite redox system, 25-40° C. is usually sufficient.

The header composition is added to the reactor composition over 30 to 1440 minutes. Preferably the addition rate will be sufficiently timed so that at least 80% of the total monomer has been reacted when the addition is completed.

Optionally, the reaction mixture will be further heated for up to 48 hours. Preferably, both the header and reactor contents will be degassed to remove oxygen. This can be done by sparging the contents with nitrogen or argon or some other suitably inert gas, or by subjecting the contents to freeze-pump-thaw cycles followed by blanketing the contents with nitrogen or argon. The nanogel may further be purified by dialysis, ultrafiltration, diafiltration, or treatment with ion exchange resins.

Those of ordinary skill in the art will recognize that even when the practice of the invention is confined, for example, to certain nanogels there are numerous methods of nanogel preparation and dispersion that will yield the nanogels with the desired characteristics. Thus any method resulting in a nanogel species with the desired characteristics is suitable for preparation of the polymer networks and biological agent compositions thereof. A useful summary of some of these methods is given in Advances in Colloid and Interface Science 1999, 80, 1-25. These methods include inverse emulsion and microemulsion techniques, such as those described in Journal of the American Chemical Society 2002, 124, 15198-15207, Molecular Pharmaceutics 2005, 2, 83-91, or in U.S. Pat. No. 5,874,111, Batch solution polymerization such as described in Macromolecular Symposia 1995, 93, 293-300 and in Macromolecules 2002, 35, 3668-3674, and high dilution crosslinking methods, such as those described in U.S. Pat. No. 6,890,703.

The following examples are provided to illustrate the invention.

All reagents were obtained from Aldrich except where noted. Quasi-elastic light scattering measurements were obtained using a Nano ZS Model ZEN3600 (Malvern Instruments) with a 633 nm laser utilizing a backscatter detector at 173 degrees. The samples were run at a concentration of 0.1-0.4% in phosphate buffered saline. Size exclusion chromatography was performed in 1,1,1,3,3,3-hexafluoro-2-propanol at 45.0° C. using two Polymer Laboratories Mixed-C columns. The Instrument, which is described in T. H. Mourey, T. G. Bryan, J. Chromatogr., 964, 169-178 (2002), was equipped with two-angle elastic light scattering (PD2020, Precision Detectors), differential viscometry (Viscotek Model H502A), spectrophotometric and differential refractive index detection. Molecular weight distributions were also measured on some materials in aqueous phosphate buffered saline (0.137 M NaCl, 0.0027M KCl, 0.01M Na₂PO₄, 0.002M KH₂PO₄) at 25.0° C. using two PSS Suprema columns. Absolute molecular weights were measured in PBS by two-angle light scattering detection.

### EXAMPLE 1

### Preparation of Amine-Terminated Poly(Ethylene Glycol) Macromonomer

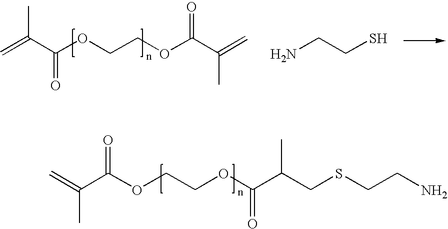

Polyethyleneglycol dimethacrylate (Aldrich, Mn=875) 335 g was mixed with 100 ml of methanol and treated with cysteamine (Aldrich, MW 77) 5.8 g and diisopropylethylamine (Hunigs base) and was stirred at RT for 2 days and concentrated using a rotary evaporator. The residue was taken up in 1 L of ethyl acetate and extracted with aqueous 10% HCl. The aqueous layer was collected and made basic by the addition of 50% aqueous sodium hydroxide followed by extraction with ethyl acetate. The organic layer was dried over MgSO₄, filtered and concentrated. The residue was taken up in anhydrous diethyl ether and treated with gaseous HCl and allowed to stand. The ether was decanted to leave a dark blue oil. This material washed with fresh diethyl ether, which was decanted. The dark blue oil was concentrated using a rotary evaporator to give 37 g of the desired product as the hydrochloride salt.

¹H-NMR (300 MHZ, CDCl₃): D 1.18 (d, 3H), 1.93 (bs, 3H), 2.04 (bs, 2H), 2.43-2.77 (bm, 7H), 3.6-3.7 (vbs, —CH₂CH₂O—), 3.73 (bt, 2H), 3.29 (bt, 2H), 5.56 (bs, 1H), 6.12 (bs, 1H)

### EXAMPLE 2

### Sulfonated Methacrylic Acid Nanogel with 9.30 Mol % Crosslinker. (Nanogel 1)

A 500 ml 3-neck round bottomed flask was modified with Ace #15 glass threads at the bottom and a series of adapters allowing connection of 1/16 inch ID Teflon tubing. The flask (hereafter referred to as the “header” flask) was outfitted with a mechanical stirrer, rubber septum with syringe needle nitrogen inlet. The header flask was charged with methacrylic acid (4.88 g, 5.66×10⁻² mol), methylene bisacrylamide (1.13 g, 7.30×10⁻³ mol), poly(ethylene glycol) monomethyl ether methacrylate (11.81 g, 1.07×10⁻² mol, Mₙ=1100), potassium sulfopropyl methacrylate (0.95 g, 3.80×10⁻² mol), 2,2′-azobis(N,N′-dimethyleneisobutyramidine)dihydrochloride (0.26 g), 1N NaOH (3.96 g) and distilled water (73.80 g). A 1 L 3-neck round bottomed flask outfitted with a mechanical stirrer, reflux condensor, nitrogen inlet, and rubber septum (hereafter referred to as the “reactor”) was charged with 2,2′-azobis(N,N′-dimethyleneisobutyramidine)dihydrochloride (0.26 g), 1N NaOH (3.96 g), and distilled water (149.84 g). Both the header and reactor contents were stirred until homogeneous and were bubble degassed with nitrogen for 20 minutes. The reactor flask was placed in a thermostatted water bath at 50° C. and the header contents were added to the reactor over four hours using a model QG6 lab pump (Fluid Metering Inc. Syossett, N.Y.). When the addition was complete, a “chaser” of 2,2′-azobis(N,N′-dimethyleneisobutyramidine)dihydrochloride (0.06 g) was added and the reaction mixture was allowed to stir at 50° C. for 16 hours. The reaction mixture was the dialyzed for 48 hours using a 14K cutoff membrane in a bath with continual water replenishment. 504 g of a colorless solution of 1.98% solids was obtained. The volume median diameter was found to be 18.6 nm with a coefficient of variation of 0.3 by quasi-elastic light scattering. Size exclusion chromatography in phosphate buffered saline gave Mn=24,700, Mw=64,600, Mz=122,000, and Mw/Mn=2.61, Mz/Mw=1.88. The Φ₂ parameter was calculated to be 3.18% and the weight average degree of polymerization was calculated to be 270.

### EXAMPLE 3

### Amine Functionalized Methacrylic Acid Nanogel with 8.22 Mol % Crosslinker. (Nanogel 2)

This nanogel was prepared using the same method as described in Example 2 except that the header addition time was 2 hours and the dialysis was performed using a 3.5K cutoff membrane. The header contained methacrylic acid (3.85 g, 4.47×10⁻² mol) Divinylbenzene (0.79 g, 6.00×10⁻³ mol, mixture of isomers, 80% pure with remainder being ethylstyrene isomers), the amine-terminated poly(ethylene glycol) macromonomer of Example 1 (7.85 g, 8.00×10⁻³ mol), 2,2′-azobis(N,N′-dimethyleneisobutyramidine)dihydrochloride (0.06 g), cetylpyridinium chloride (0.31 g), distilled water (76.40 g), and 1N NaOH (3.13 g). The reactor contents were distilled water (155.11. g), 2,2′-azobis(N,N′-dimethyleneisobutyramidine)dihydrochloride (0.06 g), cetylpyridinium chloride (0.94 g), and 1N NaOH (3.13 g). The “chaser” consisted of 2,2′-azobis(N,N′-dimethyleneisobutyramidine)dihydrochloride (0.04 g). 187.4 g of a clear dispersion of 3.48% solids was obtained. The volume median diameter was found to be 22.4 nm with a coefficient of variation of 0.45 by quasi-elastic light scattering. Size exclusion chromatography in hexafluoro-2-propanol gave Mn=206,000, Mw=1,113,000, and Mz=2,62,000, Mw/Mn=5.41, Mz/Mw=2.30 and [η]=0.288 dL/g in HFIP. The Φ₂ parameter was calculated to be 31.54% and the weight average degree of polymerization was calculated to be 5230.

### EXAMPLE 4

### Amine Functionalized Hydroxyethyl Methacrylate Nanogel with 1.94 Mol % Crosslinker (Nanogel 3)

This nanogel was prepared using the same method as described in Example 2 except that the header addition time was 2 hours. The header contained hydroxyethyl methacrylate (3.91 g, 3.00×10⁻² mol), methylenebisacrylamide (0.12 g, 7.46×10⁻⁴ mol), the amine-terminated poly (ethylene glycol) macromonomer of Example 1 (7.48 g, 7.57×10⁻³ mol), 2,2′-azobis(N,N′-dimethyleneisobutyramidine)dihydrochloride (0.12 g), and distilled water (72.11 g). The reactor contents were composed of distilled water (146.40 g), 2,2′-azobis(N,N′-dimethyleneisobutyramidine)dihydrochloride (0.12 g). The “chaser” consisted of 2,2′-azobis(N,N′-dimethyleneisobutyramidine)dihydrochloride (0.04 g). 252.0 g of a clear dispersion of 3.46% solids was obtained. The volume median diameter was found to be 25.8 nm with a coefficient of variation of 0.3 by quasi-elastic light scattering. Size exclusion chromatography in hexafluoro-2-propanol gave Mn=83,800, Mw=383,000, Mz=1,070,000, Mw/Mn=4.57, Mz/Mw=2.79 and [η]=0.452 dL/g in HFIP. The Φ₂ parameter was calculated to be 7.07% and the weight average degree of polymerization was calculated to be 1278

### EXAMPLE 5

### Hydroxyethyl Methacrylate Nanogel with 1.98 Mol % Crosslinker (Nanogel 4)

This nanogel was prepared using the same method as described in Example 2 except that the header addition time was 2 hours. The header contained hydroxyethyl methacrylate (3.91 g, 3.00×10⁻² mol), methylenebisacrylamide (0.12 g, 7.46×10⁻⁴ mol), poly(ethylene glycol)monomethyl ether methacrylate (7.48 g, 6.80×10⁻³ mol), 2,2′-azobis(N,N′-dimethyleneisobutyramidine)dihydrochloride (0.12 g), and distilled water (72.11 g). The reactor contents were composed of distilled water (146.40 g), and 2,2′-azobis(N,N′-dimethyleneisobutyramidine)dihydrochloride (0.12 g). The “chaser” consisted of 2,2′-azobis(N,N′-dimethyleneisobutyramidine)dihydrochloride (0.04 g). 252.0 g of a clear dispersion of 3.46% solids was obtained. The volume average diameter was found to be 15.7 nm with a coefficient of variation of 0.3 by quasi-elastic light scattering. Size exclusion chromatography in hexafluoro-2-propanol gave Mn=53,400, Mw=171,000, Mz=325,000, Mw/Mn=3.20, Mz/Mw=1.90 and [η]=0.830 dL/g in HFIP. The Φ₂ parameter was calculated to be 14.01% and the weight average degree of polymerization was calculated to be 559.

### EXAMPLE 6

### Hydroxyethyl Methacrylate Nanogel with 7.70 Mol % Crosslinker (Nanogel 5)

This nanogel was prepared using the same method as described in Example 2 except that the header addition time was 2 hours. The header contained hydroxyethyl methacrylate (3.80 g, 2.92×10⁻² mol), methylenebisacrylamide (0.46 g, 2.98×10⁻³ mol), poly(ethylene glycol)monomethyl ether methacrylate (7.25 g, 6.59×10⁻³ mol), 2,2′-azobis(N,N′-dimethyleneisobutyramidine)dihydrochloride (0.12 g), and distilled water (72.11 g). The reactor contents were composed of distilled water (146.40 g), and 2,2′-azobis(N,N′-dimethyleneisobutyramidine)dihydrochloride (0.12 g). The “chaser” consisted of 2,2′-azobis(N,N′-dimethyleneisobutyramidine)dihydrochloride (0.04 g). 252.0 g of a clear dispersion of 3.46% solids was obtained. The volume median diameter was found to be 21.8 nm with a coefficient of variation of 0.3 by quasi-elastic light scattering. Size exclusion chromatography in hexafluoro-2-propanol gave Mn=117,000, Mw=283,000, Mz=555,000, Mw/Mn=2.42, Mz/Mw=1.96 and [η]=0.670. The Φ₂ parameter was calculated to be 8.66% and the weight average degree of polymerization was calculated to be 952.

### EXAMPLE 7

### Hydroxyethyl Methacrylate Nanogel with 11.19 Mol % Crosslinker (Nanogel 6)

This nanogel was prepared using the same method as described in Example 2 except that the header addition time was 2 hours. The header contained hydroxyethyl methacrylate (3.80 g, 2.92×10⁻² mol), methylenebisacrylamide (0.46 g, 4.48×10⁻³ mol), poly(ethylene glycol)monomethyl ether methacrylate (7.48 g, 6.38×10⁻³ mol), 2,2′-azobis(N,N′-dimethyleneisobutyramidine)dihydrochloride (0.12 g), and distilled water (72.11 g). The reactor contents were composed of distilled water (146.40 g), and 2,2′-azobis(N,N′-dimethyleneisobutyramidine)dihydrochloride (0.12 g). The “chaser” consisted of 2,2′-azobis(N,N′-dimethyleneisobutyramidine)dihydrochloride (0.04 g). 255 g of a clear dispersion of 3.05% solids was obtained. The volume median diameter was found to be 27.7 nm with a coefficient of variation of 0.4 by quasi-elastic light scattering. Size exclusion chromatography in 1,1,1,3,3,3-hexafluoro-2-propanol gave Mn=533,000, Mw=1,265,000, Mz=3,800,000, Mw/Mn=2.37, Mz/Mw=3.00 and [η]=0.773 dL/g in HFIP. The Φ₂ parameter was calculated to be 19.93% and the weight average degree of polymerization was calculated to be 4,399.

Quasi-elastic light scattering was performed on the nanogel at a series of temperatures. The data do not exhibit an abrupt decrease in size indicative of a lower critical solution temperature in the temperature range examined.

TABLE 1
Hydrodynamic diameter of Nanogel 6 as a function of temperature.

| Temperature | Hydrodynamic |
|---|---|
| (° C.) | diameter (nm) |
| 25 | 28.9 |
| 34 | 36.3 |
| 43 | 38.6 |
| 52 | 31.9 |
| 61 | 33.4 |
| 70 | 33.3 |

### EXAMPLE 8

### Sulfonated Methacrylic Acid Nanogel with 9.30 Mol % Crosslinker. (Nanogel 7)

This nanogel was prepared using the same method as described in Example 2 except that the header addition time was 2 hours. The header contained methacrylic acid (4.88 g, 5.66×10⁻² mol) methylene bisacrylamide (1.13 g, 7.30×10⁻³ mol), poly(ethylene glycol)monomethyl ether methacrylate (11.81 g, 1.07×10⁻² mol), potassium sulfopropyl methacrylate (0.94 g, 3.81×10⁻³ mol), 2,2′-azobis(N,N′-dimethyleneisobutyramidine)dihydrochloride (0.26 g), distilled water (73.80 g), and 1N NaOH (3.96 g). The reactor contents were composed of distilled water (149.84. g), 2,2′-azobis(N,N′-dimethyleneisobutyramidine)dihydrochloride (0.26 g), and 1N NaOH (3.96 g). The “chaser” consisted of 2,2′-azobis(N,N′-dimethyleneisobutyramidine)dihydrochloride (0.06 g). 441 g of a clear dispersion of 4.05% solids was obtained. The volume median diameter was found to be 23.8 nm with a coefficient of variation of 0.3 by quasi-elastic light scattering. Size exclusion chromatography in phosphate buffered saline gave Mn=98,700, Mw=406,500, and Mz=976,500, Mw/Mn=4.12, Mz/Mw=2.40. The Φ₂ parameter was calculated to be 9.56% and the weight average degree of polymerization was calculated to be 1701.

### EXAMPLE 9A

### Sulfonated Methacrylic Acid Nanogel with 6.21 Mol % Crosslinker. (Nanogel 8)

This nanogel was prepared using the same method as described in Example 2 except that the header addition time was 2 hours. The header contained methacrylic acid (5.06 g, 5.88×10⁻² mol) methylene bisacrylamide (0.75 g, 4.86×10⁻³ mol), poly(ethylene glycol)monomethyl ether methacrylate (12.00 g, 1.09×10⁻² mol), potassium sulfopropyl methacrylate (0.94 g, 3.81×10⁻³ mol), 2,2′-azobis(N,N′-dimethyleneisobutyramidine)dihydrochloride (0.26 g), distilled water (73.71 g), and 1N NaOH (4.11 g). The reactor contents were composed of distilled water (149.65. g), 2,2′-azobis(N,N′-dimethyleneisobutyramidine)dihydrochloride (0.26 g), and 1N NaOH (4.11 g). The “chaser” consisted of 2,2′-azobis(N,N′-dimethyleneisobutyramidine)dihydrochloride (0.06 g). 434.6 g of a clear dispersion of 3.87% solids was obtained. The volume median diameter was found to be 22.0 nm with a coefficient of variation of 0.3 by quasi-elastic light scattering. Size exclusion chromatography in phosphate buffered saline gave Mn=60,100, Mw=186,500, and Mz=403,000, Mw/Mn=3.10, Mz/Mw=2.16. The Φ₂ parameter was calculated to be 5.50% and the weight average degree of polymerization was calculated to be 779.

### EXAMPLE 9B

### Attachment of a Near-Infrared Dye to Nanogel 1

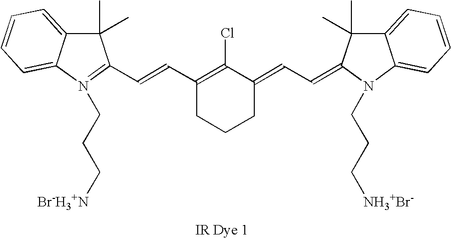

Two dye solutions were prepared in by dissolving IR dye 1 (10.46 mg (40 μmol), and 26.15 mg (100 μmol)) in 1-2 ml DMF. Similarly, solutions of the nanogel were prepared by dissolving freezedried Nanogel 1 (each 0.067 g, 200 μmol) in 1˜2 ml of DMF. 0.2 ml of 0.45M HBTU (O-Benzotriazole-N,N,N′,N′-tetramethyl-uronium-hexafluoro-phosphate, obtained from Applied Biosystems) was added into each solution of nanogel, which was then stirred for 5 minutes. 0.25 ml of 2M diisopropylethylamine (DIEA) was then added into the nanogel followed by stirring for one minute. Finally, the activated solution of nanogel were transferred into the dye solutions, which were then shaken for 4-6 hours at room temperature. Each nanogel/dye reaction solution was then poured into ˜10 ml water, and was loaded into a Centriprep® YM-30 filter cartridge (Micon Bioseparations) with a molecular weight cutoff of 30,000 Daltons and was centrifuged at 3300 RPM until ˜75% of the liquid had diffused through the membrane. The concentrate above the membrane was then re-diluted with water and the process was repeated until the permeate was colorless.

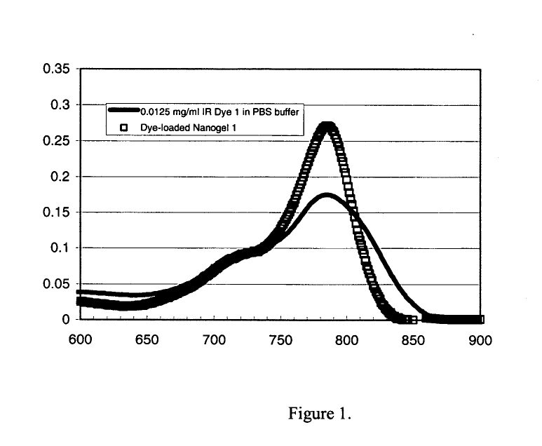

The free amount of dye was estimated by preparing a standard UV-V is absorbance curve using dye solutions of known concentrations. The absorbance value of a 10 ml of the filtration solution after coupling reaction which contains all of the non-conjugated dye was measured and the amount of free dye was quantified using the standard absorbance curve. The conjugate yield was estimated to be 98% for the case with 10.46 mg of IR dye 1. UV/V is analysis of the dye-nanogel conjugates showed a λₘₐₓ of 784 nm with a small shoulder at ˜725 nm (see FIG. 1). This shoulder, which is indicative of the presence of a nonemissive aggregate, was relatively small, and was no more pronounced with respect to the main peak than on the spectrum of the IR dye free in aqueous solution. This illustrates the surprising result that even at relatively high loadings, dyes attached to this particle show relatively little aggregation. Analysis of the conjugate by quasi-elastic light scattering showed an increase in volume average diameter from 18.6 to 24.1 nm.

### EXAMPLE 10

### Cytotoxicity Study of Nanogels Using Cell Viability Assay

Human umbilical endothelia cells (HUVEC, purchased from Cascade Biologics, Inc. (Portland, Oreg.)) were maintained in Medium 200 containing 2% fetal bovine serum and antibiotics. HUVEC (2×104 cells/well) were plated on 96 wells plate in the complete medium. Next day after the plate wells were washed with serum free medium, nanogels were added at the concentration as indicated in Table 2. 24 hours later the cytotoxicity was determined using the CellTiter-Glo® Luminescent cell viability assay kit (Promega Corp., Madison, Wis.). This assay is a homogeneous method of determining the number of viable cells in culture based on quantitation of the ATP present, an indicator of metabolically active cells. CellTiter-Glo® reagent was mixed with phosphate buffered saline pH 7.4 at ratio of 1 to 1, than added to the cell well. Luminescence of each well was measured with Fluster OPTIMA plate reader (BMG LABTECH). Results (see Table 2) are expressed as mean value of three duplicate determinations. Nanogel 1 is a duplicate batch as that described in Example 2.

TABLE 2
Cell viability assay results for Nanogels 1, 3, 4, and 5.

| Sample | % |
|---|---|
| Analyzed | viability* |
| Control | 100.00 |
| Nanogel 4 (0.2 mg/ml) | 98.71 |
| Nanogel 4 (0.02 mg/ml) | 112.58 |
| Nanogel 5 (0.2 mg/ml) | 96.89 |
| Nanogel 5 (0.02 mg/ml) | 112.69 |
| Nanogel 3 (0.2 mg/ml) | 99.36 |
| Nanogel 3 (0.02 mg/ml) | 124.45 |
| Nanogel 1 (duplicate batch) | 81.33 |
| (0.2 mg/ml) | |
| Nanogel 1 (duplicate batch) | 80.69 |
| (0.02 mg/ml) | |
| Silica (0.2 mg/ml) | 26.20 |
| Silica (0.02 mg/ml) | 24.05 |

*% viability = (sample OD/control OD) * 100

### EXAMPLE 11

### Attachment of Fluorescent Dye to Amine Containing Nanogel (Nanogel 3)

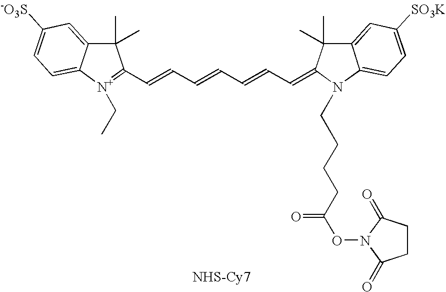

A NHS-cy7 dye stock solution was prepared by dissolving 1 mg of NHS-cy7 dye (Purchased from GE Healthcare, Buckinghamshire, UK) in 1 mL of DMF. Aliquots of cy7 stock solution was added to a PBS buffer solution containing 0.05% (w/v) nanogel to a final volume of 10 mL. The mixture was stirred for 3 hours covered from room light, then filtered through Centriprep® YM-30 (30,000 MW cutoff) filters, and washed with PBS buffer, retaining the filtrate, until the filtrate is clear. The volume and absorbance of the filtrates were measured to determine the amount of cy7 attached to nanogels. The results are shown in Table 3.

TABLE 3

| Sample | mg Cy7/mg |
|---|---|
| I.D. | nano |
| 11-1 | 0.045 |
| 11-2 | 0.088 |
| 11-3 | 0.166 |
| 11-4 | 0.237 |

### EXAMPLE 12

### Attachment of Biotin-PEG to Nanogels

Portions of Biotin-mPEG-NHS (purchased from Nektar) (3.2 mg, 6.4 mg, and 12.8 mg) were added to 3 different vials containing 10 mL of 0.05% nanogel in PBS buffer. The mixture was stirred for 2 hours, filtered through 30,000 mw filters, discarding filtrate, and washing with PBS buffer. The final volume after filtration for all samples was brought to 4 mL with PBS buffer.

The amount of Biotin attached to nanogels was determined by a ligand displacement assay using HABA/Avidin (purchased from Pierce). In a typical assay, HABA/Avidin was dissolved in a vial with 100 μL PBS buffer, followed by 800 μL of PBS buffer in a 1 cm cuvette. The sample was mixed well and the absorbance at 500 nm was recorded. Then a 100 mL of biotin attached nanogel was added to the HABA/Avidin solution, the absorbance at 500 nm was recorded again, and the difference between the two measurements was used to calculate the amount of biotin attached to nanogel samples. In sample 12-1, NHS-cy7 was added to the solution and stirred in the dark for 2 hours. The unattached dye was filtered out with a YM-30 filter until filtrate is clear. The volume and absorbance of filtrate was measured to determine the amount of cy7 attachment. The results were shown in Table 4.

TABLE 4

| | Abs. At | | % of biotin | μg of cy7/mg |
|---|---|---|---|---|
| | 500 nm | Difference | attachment | nanogel |
| control | 0.852 | 0 | | |
| 12-1 | 0.727 | 0.0398 | 73.2% | 210 |
| 12-2 | 0.691 | 0.0875 | 80.4% | |
| 12-3 | 0.573 | 0.1857 | 85.3% | |

The results in examples 11-12 demonstrated that the nanogels of this invention can be used for the attachment of payload of imaging contrast agent or therapeutics in a covalent manner and a bio-targeting moiety can also be attached to the nanogel surface for bio-target recognition.

### EXAMPLE 13

### Stability of Nanogels in 1.5M NaCl

Quasi-elastic light scattering measurements were performed on Nanogels 2-7 in 1.5M NaCl solution. The volume average diameters, which are all very similar to the values in PBS buffer, are reported below in Table 5. This example illustrates the colloidal stability of the nanogels in concentrated electrolyte solution.

TABLE 5
QELS results for nanogels in PBS buffer and in 1.5 M NaCl.

| | Dᵥ in PBS | Dᵥ in 1.5 M |
|---|---|---|
| | Buffer (nm) | NaCl (nm) |
| Nanogel 2 | 22.4 | 22.2 |
| Nanogel 3 | 25.8 | 20.4 |
| Nanogel 4 | 15.7 | 16.5 |
| Nanogel 5 | 21.8 | 22.0 |
| Nanogel 6 | 27.7 | 25.8 |
| Nanogel 7 | 23.8 | 23.3 |

### EXAMPLE 14

### Protein Binding of Nanogels in Phosphate-Buffered Saline

Nanogels 4, 6, 8, 12 and 13 were individually mixed with an equal amount by weight of bovine serum albumin (BSA) at a concentration of 1.5 mg/mL in phosphate buffered saline. The mixtures were examined by size-exclusion chromatography (SEC) in phosphate buffered saline on two PSS Suprema mixed-bed columns at 30° C. and the resulting chromatograms were compared to those of the nanogels and BSA alone. BSA exhibits a sharp, distinguishable monomer peak, a characteristic high-molecular-weight shoulder from dimers and larger species, and exhibits strong ultraviolet (UV) absorption at 270 nm with an on-line UV detector. These characteristics make the chromatographic peak of BSA clearly distinguishable from those of nanogels, which are not observed with a UV detector at 270 nm and are only detected by differential refractive index detection. The BSA peak appeared unchanged compared to BSA alone in each of the sample mixtures examined. The chromatographic evidence is consistent with no enlargement in size of BSA, such as would occur with strong binding of nanogels to BSA molecules.

### COMPARATIVE EXAMPLE 1

### Attempt at Preparation of Sulfonated Methacrylic Acid Nanogel of Example 7 by Batch Reaction

A 1 L 3-neck round bottomed flask outfitted with a reflux condenser, nitrogen inlet, and mechanical stirrer was charged with methacrylic acid (4.88 g, 5.66×10⁻² mol) methylene bisacrylamide (1.13 g, 7.30×10⁻³ mol), poly(ethylene glycol)monomethyl ether methacrylate (11.81 g, 1.07×10⁻² mol), potassium sulfopropyl methacrylate (0.94 g, 3.81×10⁻³ mol), distilled water (223.65 g), and 1N NaOH (7.922 g). The flask was placed in a thermostatted water bath at 50° C. When the contents had reached 50° C., 2,2′-azobis(N,N′-dimethyleneisobutyramidine)dihydrochloride (0.52 g) was added. Within two hours the reaction contents had gelled.

The invention has been described in detail with particular reference to certain preferred embodiments thereof, but it will be understood that variations and modifications can be effected within the spirit and scope of the invention.

1. A nanogel comprising a water-compatible, swollen, branched polymer network of repetitive, crosslinked, ethylenically unsaturated monomers of Formula I:
(X)m-(Y)n-(Z)o Formula I
Wherein:
X is a water-soluble monomer containing ionic or hydrogen bonding moieties;
Y is a water-soluble macromonomer containing repetitive hydrophilic units bound to a polymerizeable ethylenically unsaturated group;
Z is a multifunctional crosslinking monomer;
m ranges from 50-90 mol %;
n ranges from 2-30 mol %; and
o range from 1-15 mol %.

2. The nanogel of claim 1 wherein m ranges from 60-80 mol %, n ranges from 10-20 mol %, and o ranges from 2-9 mol %.

3. The nanogel of claim 1 wherein X is a water-soluble monomer containing ionic or exchangeable proton-containing moieties.

4. The nanogel of claim 1 wherein X comprises at least one member selected from the group consisting of alcohols, primary and secondary amines, primary amides, secondary amides, carboxylic acids, carbamates, imides, ureas, phosphonic acids, sulfonic acids, sulfinic acids, and any other unit which contains a heteroatom (N,O,S,P)-hydrogen bond.

5. The nanogel of claim 1 wherein X is represented by Formula II or Formula III: 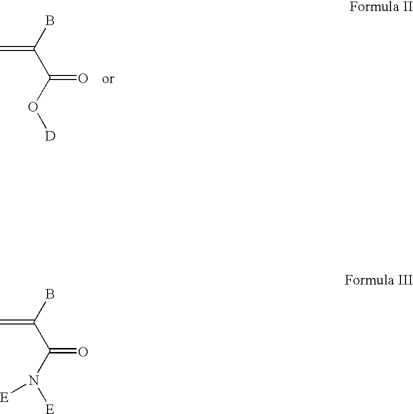
Wherein
B is H or CH₃;
D is H, a nonionic unit with a hydrogen bonding moiety and containing no more than three carbons, or an ionic unit comprised of up to six carbons; and
E is H, or CH₃.

6. The nanogel of claim 1 wherein X is methacrylic acid, acrylic acid, acrylamide, methacrylamide, aminopropyl methacrylamide hydrochloride, sulfopropyl methacrylate, hydroxyethyl acrylate or hydroxyethyl methacrylate, N-methyl acrylamide, or N,N-dimethylacrylamide.

7. The nanogel of claim 1 wherein X is x-hydroxyethyl methacrylate or methacrylic acid.

8. The nanogel of claim 1 wherein X has a calculated log P value of 0.4 or less.

9. The nanogel of claim 1 wherein Y is a water-soluble macromonomer with a molecular weight of from 200 to 20,000, and is comprised of repetitive water-soluble units.

10. The nanogel of claim 1 wherein water-soluble macromonomer Y is a poly(ethylene glycol) macromonomer.

11. The nanogel of claim 10 wherein Y is selected from the group consisting of poly(ethylene glycol)acrylate, poly(ethylene glycol)methacrylate, N-poly(ethylene glycol)acrylamide, N-poly(ethylene glycol)methacrylamide, and a poly(ethylene glycol) macromonomer with a styrenic terminus.

12. The nanogel of claim 1 wherein Y is a poly(ethylene glycol) macromonomer backbone with a radical polymerizeable group at one end of said macromonomer backbone and a different reactive chemical functionality at the other end of said macromonomer backbone, according to Formula I: 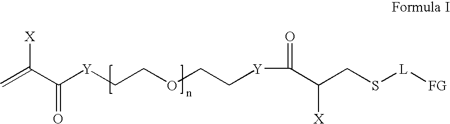
wherein:
X is CH₃, CN or H;
Y is O, NR₁, or S;
L is a linking group or spacer;
FG is a functional group excluding alkoxy silanes;
n is greater than 4 and less than 1000; and
wherein R₁ and R₂ are independently selected from substituted or unsubstituted alkyl, aryl, or heteroyl.

13. The nanogel of claim 1 wherein Z is methylenebisacrylamide, N,N′-(1,2-dihydroxyethylene)bisacrylamide, methylenebismethacrylamide divinylbenzene, or ethylene glycol dimethacrylate.

14. The nanogel of claim 1 wherein Z is difunctional, trifunctional, or tetrafunctional and has a molecular weight of less than 300 Daltons.

15. The nanogel of claim 1 wherein at least 90% of the total of X, Y, and Z is highly hydrophilic or water-soluble.

16. The nanogel of claim 1 wherein said nanogel has a volume-median hydrodynamic diameter of from 10 to 50 as determined by quasi-elastic light scattering in phosphate buffered saline (137 mM NaCl, 2.7 mM KCl, 10 mM Na₂HPO₄, 2 mM KH₂PO₄ at pH 7.4.).

17. The nanogel of claim 1 wherein said nanogel has a weight average molecular weight of from 15,000 to 6,000,000 as measured by static light scattering or by size exclusion chromatography.

18. The nanogel of claim 1 wherein the weight average degree of polymerization of said nanogel is from 50 to 86,000.

19. The nanogel of claim 1 wherein said nanogel has a φ₂ parameter of from 0.01 to 0.30 in water.

20. The nanogel of claim 1 wherein said nanogel is stable in 1.5M NaCl.

21. The nanogel of claim 1 wherein said nanogel has an intrinsic viscosity of from 0.40 dL/g to 0.85 dL/g.

22. The nanogel of claim 1 wherein said nanogel experiences a net increase of hydrodynamic diameter upon raising the temperature from 25° C. to 80° C.

23. The nanogel of claim 1 wherein the degree of polymerization of said nanogel is from 20 to 1500.

24. The nanogel of claim 1 wherein the deswelling ratio of said nanogel is from 0.02 to 0.2.

25. The nanogel of claim 1 wherein said nanogel is substantially serum protein non-adsorbent to bovine serum albumin (BSA).

26. The nanogel of claim 1 wherein said nanogel comprises further comprises at least one carried compound associated with said nanogel.

27. The nanogel of claim 26 wherein said at least one carried compound associated with said nanogel is a biological, pharmaceutical or diagnostic compound.

28. The nanogel of claim 26 wherein said at least one carried compound associated with said nanogel is non-covalently associated.

29. The nanogel of claim 26 wherein said at least one carried compound associated with said nanogel is covalently associated.

30. The nanogel of claim 29 wherein said covalent association is formed to X, Y, or Z and polymerized directly into the nanogel during the nanogel preparation.

31. The nanogel of claim 26 wherein said at least one carried compound associated with said nanogel is a dye.

32. The nanogel of claim 26 wherein said at least one carried compound associated with said nanogel is a dye and a targeting moiety.

33. A method for preparing a nanogel comprising:
a. preparing a header composition of a mixture of monomers X, Y, and Z, and a first portion of initiators in water, wherein X is a water-soluble monomer containing ionic or hydrogen bonding moieties, Y is a water-soluble macromer containing repetitive hydrophilic units bound to a polymerizeable ethylenically unsaturated group, and Z is a multifunctional crosslinking monomer;
b. preparing a reactor composition of a second portion initiators, surfactant, and water sufficient to afford a composition of 1-10% w/w of monomers X, Y, and Z,
c. bringing said reactor composition to the polymerization temperature,
d. holding said reactor composition at said polymerization temperature for the duration of the reaction, and
e. adding said header composition to said reactor composition over time to form a reaction mixture;
wherein said nanogel comprises a water-compatible, swollen, branched polymer network of repetitive, crosslinked, ethylenically unsaturated monomers of Formula I:
(X)m-(Y)n-(Z)o Formula I
wherein:
m ranges from 50-90 mol %;
n ranges from 2-30 mol %; and
o range from 1-15 mol %.

34. The method of claim 33 wherein said mixture of monomers X, Y, and Z comprises 50-90 mol % of X, 2-30 mol % of Y, and 1-20 mol % of Z.

35. The method of claim 33 wherein said initiator is a water-soluble polymerization initiator.

36. The method of claim 35 wherein said water-soluble polymerization initiator is a water-soluble azo initiator.

37. The method of claim 33 wherein said initiator is a redox initiator.

38. The method of claim 33 wherein said initiator is a two component initiator, wherein one component of said two component initiator is included in said header composition and the other component of said two component initiator is included in said reactor composition, such that free radicals are steadily generated as said header composition and said reactor composition are combined.

39. The method of claim 33 wherein said initiator is a water-soluble photoinitiator.

40. The method of claim 33 wherein said time for adding said header composition to said reactor composition is from 30 to 1440 minutes.

41. The method of claim 33 wherein said adding said header composition to said reactor composition over time occurs at an addition rate sufficient timed so that at least 80% of the total monomer has been reacted when said adding is completed.

42. The method of claim 33 wherein said header composition further comprises surfactant.

43. The method of claim 33 wherein further comprising heating said reaction mixture for up to 48 hours.

44. The method of claim 33 further comprising purifying said reaction mixture by dialysis, ultrafiltration, diafiltration, or treatment with ion exchange resins.

45. The method of claim 33 further comprising degassing said header composition and said reactor composition to remove oxygen.

46. The method of claim 45 wherein said degassing is by sparging the contents with nitrogen or argon or some other suitably inert gas, or by subjecting the contents to freeze-pump-thaw cycles followed by blanketing the contents with nitrogen or argon.
